(19) United States
(12) Patent Application Publication
(10) Pub. No.: US 2023/0381531 A1
(43) Pub. Date: Nov. 30, 2023

(54) INGESTIBLE RED, INFRARED, NEAR-INFRARED LIGHT EMITTING CAPSULE FOR HUMAN AND ANIMAL THERAPEUTIC USE

(71) Applicant: DeBeer Medical, Inc., Delray Beach, FL (US)

(72) Inventors: Brad DeBeer, Golden, CO (US); Brian DeBeer, Delray Beach, FL (US)

(21) Appl. No.: 17/964,148

(22) Filed: Oct. 12, 2022

Related U.S. Application Data
(60) Provisional application No. 63/346,692, filed on May 27, 2022.

(51) Int. Cl. A61N 5/06

(52) U.S. Cl. CPC A61N 5/0603; A61N 2005/0659; A61N 2005/0663; A61N 2005/0652; A61N 2005/0626

(57) ABSTRACT

This invention is directed to a device that introduces light therapy to the inter-lumen of the gastrointestinal tract. The device is an ingestible light-emitting capsule for human and animal therapeutic use. The invention provides a convenient, efficient, and safe way to treat the cellular causative mechanisms and ease the symptoms of gastric gastrointestinal, and neurodegenerative disease. Additionally, it provides a means for DNA repair, protection, and enhancement of the gut microbiome.

### CROSS-REFERENCE TO RELATED APPLICATIONS

This invention claims priority to provisional application No. 63/346,692, filed on May 27, 2022.

### BACKGROUND OF THE INVENTION

A healthy gastrointestinal function requires a lot of different inputs for optimal health; examples include non-inflammatory diets, medications, and cleanses. The former examples, however, are widely used; but with very little success in overcoming GI dysfunction such as Crohn's, irritable bowel syndrome (IBS), ulcers, and ulcerative colitis. Additionally, current literature suggests that altered gut and brain connections are a leading cause of neurodegeneration, such as Parkinson's and Alzheimer's.

Current literature widely supports using blue, green, red, infrared, and near-infrared light to increase energy and support repair. Light therapy has been shown clinically and in practice to decrease the symptoms of Chron's, IBS, leaky gut, ulcers, and ulcerative colitis by affecting the cellular mechanism that initiates and causes these disorders. Among other neurological enhancements, light therapy improves energy production and the removal of inflammatory mediators, thus increasing the stability of cell junctions and mucus membranes, thereby improving digestion and absorption, improving serotonin release in the gut, and enhancing the microbiome, supporting the diversity and sustainability of the microflora, which restores vagus nerve signaling and improves the Gut-Brain Axis.

Recent studies have found that shining a variety of individual spectrums of light, including blue, green, red, and near-infrared, on the skin over a problem area reduces gut dysfunction. The GI tract, however, is very long, and the area exposed to the light from outside the body is minimal. Thus, there is a need to provide a solution that will allow a larger area of the GI tract to be exposed to light therapy. The invention described herein meets the need by providing an ingestible capsule that will supply the entire GI tract with light treatment, thereby increasing energy, supporting the microbiome, and helping the patient achieve their goals. In addition to the disorders described above, the invention will be helpful in other known and unknown forms of diseases.

### BRIEF SUMMARY OF THE INVENTION

The invention is an ingestible device comprising a capsule that houses a plurality of light sources, a controller board, and a battery. The capsule is an ingestible capsule of the correct size for oral ingestion by a human or animal patient. The capsule is designed to pass through the GI tract safely and comfortably without releasing any drugs or chemicals into the body. The capsule is made from biocompatible materials approved for human or animal ingestion. The capsule is made to withstand the GI environment and the peristaltic movement of the GI tract.

The capsule houses an array of light sources; non-limiting examples include light-emitting diodes, laser-emitting diodes, and fiber optics. Non-limiting examples of the wavelengths of light emitted include visible, blue, green, red, infrared, near-infrared wavelengths, or any other wavelength found beneficial. The enclosed battery system will be capable of powering the entire unit for the duration of the treatment.

The light sources and battery are coupled with a controller board which will provide any of the following non-limiting functions: (1) on/off switch, (2) timer, (3) selecting for the spectrum or wavelength of the light emitted, 4) set the intensity of light emitted, or any other function desired. Additionally, the controller board could be programmable to provide any combination of the previous features, thus allowing the practitioner to select the most appropriate course of treatment. The controller board can be operated either manually or remotely.

For the purposes of this invention, the term light therapy encompasses a treatment modality where any part of the human body or animal body is irradiated with electromagnetic radiation. The wavelength may be of any single or combination of wavelengths selected from the electromagnetic spectrum, but preferably between 500 to 1000 nm, and more preferably between 590 to 750 nm or 850 to 950 nm, and most preferably between 600 to 700 nm. Additionally, light therapy includes non-limiting light therapy examples such as photobiomodulation (PBM), low-level light therapy (LLLT), soft laser therapy, cold laser therapy, biostimulation, photonic stimulation, low power laser therapy (LPLT), and red-light therapy (RLT) and optogenetics.

The claimed invention offers a mode of treatment that differs drastically from what currently exists. Current methods use transcutaneous irradiation, whereas the invention is designed to administer inter-lumen irradiation directly to the end organ. Moreover, the invention enables irradiation of the entire GI tract, where the whole GI tract starts from the oral cavity through the anus. Additionally, the device can be programmed to time the irradiation dosage for specific problem areas that are currently known or unknown to exacerbate GI dysfunction or neurodegeneration or weaken the Gut-Brain axis.

The invention is useful for gastrointestinal and neurodegeneration disease prevention, rehabilitation, general health, wellness, and longevity. It is believed that the invention will be useful for other novel neurological or gastrointestinal uses yet to be discovered. It also provides the patient with clinician-guided, self-administered cutting-edge gastrointestinal and neurological care that can continue to treat for multiple hours, or days, while at home, with the only restrictions being MRI/magnetic or metal detection based. Unlike most conventional infinite-like, side-effect-littered treatments for the conditions mentioned, the device allows the patient to be treated for a shorter time. It minimizes adverse mechanistic or chemical side effects.

The invention enhances the gastrointestinal, neurological, neurodegenerative, cognitive, and general medical fields. Including telemedicine and the at-home care industry. It allows patients to receive better and more effective care quickly. Treatment with the invention equates to less disease and better digestive tracts. Healthy digestive tracts lead to more happiness, better continuity of relationships, improved cognition in the elderly and post-injury population. Furthermore, it provides more options for individuals suffering from gastrointestinal, neurological, or other deficits where light therapy could be beneficial.

### BRIEF DESCRIPTION OF THE DRAWINGS

FIG. 1 is a perspective view of the invention's mode selector button.

FIG. 2 is a perspective view of the invention with a twist-on twist-off feature.

FIG. 3 is the vertical cross-section of the midline of FIG. 1.

FIG. 4 is the horizontal cross-section of the end cap.

FIG. 5 is the horizontal cross-section of the mid-line of FIG. 3.

FIG. 6 is a perspective view of the invention.

FIG. 7 is the vertical cross-section of the midline of FIG. 6.

FIG. 8 is the horizontal cross-section of the mid-line of FIG. 7.

FIG. 9 is the horizontal cross-section of the end cap of FIG. 7.

FIG. 10 is a perspective view of the invention.

FIG. 11 is the vertical cross-section of the midline FIG. 10.

FIG. 12 is the horizontal cross-section of the end cap of FIG. 11.

FIG. 13 is the horizontal cross-section of the mid-line of FIG. 11.

### DETAILED DESCRIPTION OF THE INVENTION

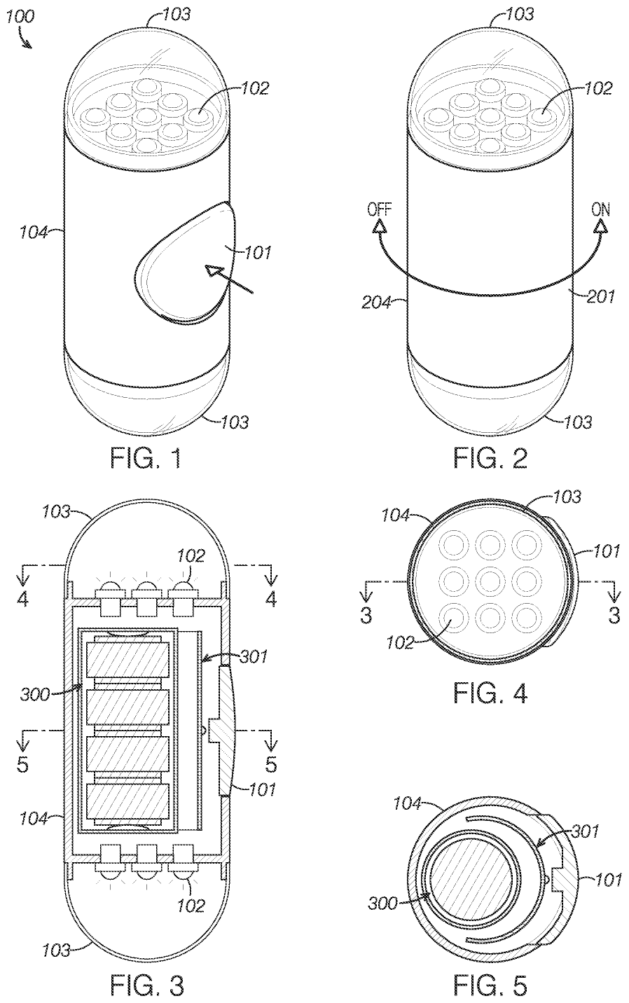

Referring to FIGS. 1-5, one embodiment of the invention comprises a capsule 100 with a first end 103 and a second end 103, and a cylinder 104 with corresponding ends to the first end 103 and the second end 103. The first and second ends, 103 and 103, are transparent, allowing light to be projected from either or both ends. Located at the corresponding ends of cylinder 104 are a plurality of light sources 102. Cylinder 104 is hollow and located within the cylinder are the batteries 300 and the controller board 301. The controller board 301 may provide any of the following non-limiting functions: (1) on/off switch, (2) timer, (3) selecting for continuous irradiation or pulsed irradiation, (4) selecting for the spectrum or wavelength of the light emitted, (5) the type of light emitted, and (6) the intensity of light emitted.

In one embodiment, the functions are pre-programmed and selected manually using button 101 or remotely wired or wireless communication with the controller board. In another embodiment, the controller board could be programmed by a wired or wireless connection. The program could then be activated using a twist switch (201). In yet another embodiment, the device can turn on or off using a button 101 or twisting 201.

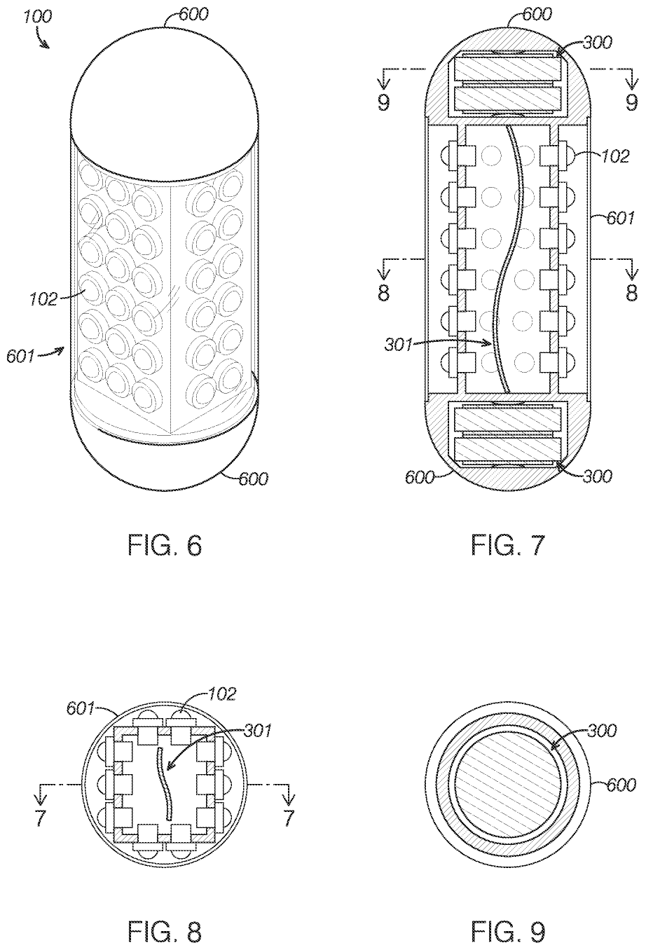

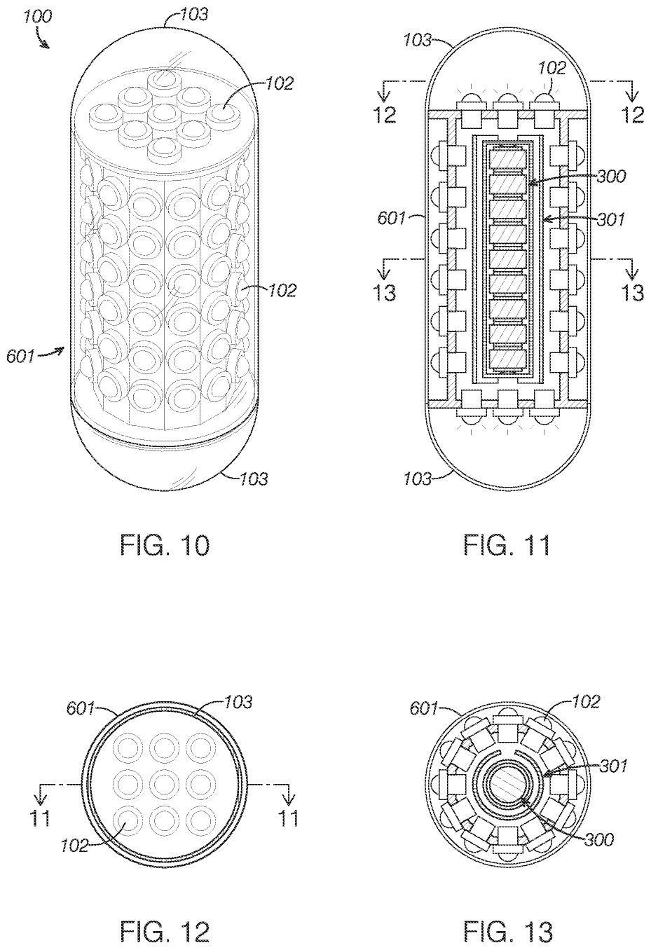

FIGS. 6-9 exhibit another embodiment of the invention. In this embodiment, cylinder 601 is transparent, and the first end 600 and second end 600 are opaque. Within cylinder 601 are an array of light sources 102. The batteries 300 are located within the first end 600 and the second end 600.

FIGS. 10-13 describe another embodiment of the invention. In this embodiment, the entire capsule 100 is transparent. It comprises a first end 103 and a second end 103, and the cylinder 601 has ends corresponding with the first and second end 103 and 103—the batteries 300 are placed vertically along the central, vertical axis of cylinder 601. Located at the corresponding ends of the cylinder 601 are a plurality of light sources 102 and within the cylinder 601 are an array of light sources 102.

The capsule 100 may be a two-piece or one-piece capsule made from any known biocompatible material on the market now or in the future. It must be air-tight and water-tight to prevent contamination from gastric juices and other bodily fluids. Additionally, it should remain sealed to avoid opening, which may harm the patient.

The light sources 102 should be small, individual and placed in varying arrays and angles. For this invention, the term light includes incoherent light, such as the light emitted by most common light sources, and coherent light such as the light emitted by a LASER. Non-limiting examples of light sources 102 include light-emitting diodes, laser-emitting diodes, and fiber optics.

The wavelength emitted can be any currently known or future known wavelength that decreases inflammation, decreases pain, improves the microbiome, or any of the benefits mentioned above. The wavelength may be of any single or combination of wavelengths selected from the electromagnetic spectrum, but preferably between 500 to 1000 nm, and more preferably between 590 to 750 nm, and most preferred between 600 to 700 nm.

A non-limiting example of the controller board 301 is a small circuit board connected to the battery and the light sources. The controller board 301 may provide the following functions: (1) on/off switch, (2) timer, (3) selecting for continuous irradiation or pulsed irradiation, (4) selecting for the spectrum or wavelength of the light emitted, (5) the type of light emitted, and (6) the intensity of light emitted. In one embodiment, the controller board 301 is pre-programmed. In another embodiment, the controller board 301 is programmed either by a wired or wireless connection. The controller board 301 may be operated manually using button 101 or remotely by wireless connection. For the purposes of this invention, the term wireless includes communications by radio frequency transmission, infrared transmission, microwave transmission, or light wave transmission. Including non-limiting examples of wireless such as radio and television broadcasting; satellite, radar, infrared, and cellular communication; WLAN (Wi-Fi), Bluetooth, ZigBee, RFID, and paging technologies.

The batteries 300 should be small enough to fit inside the housing of the capsule 100, fully self-contained, and be connected to the controller board 301 and the light sources 102. The batteries 300 should provide enough power to power the device for the time necessary for the invention to pass through the digestive tract.

For this invention, the term light includes incoherent light, such as the light emitted by most common light sources, such as incandescent light bulbs, and coherent light, such as the light emitted by a LASER. The wavelength emitted can be any currently known or future known wavelength that decreases inflammation, decreases pain, improves cell junctions or mucosa, improves the microbiome, or any of the benefits mentioned above. The wavelength may be of any single or combination of wavelengths selected from the electromagnetic spectrum, but preferably between 500 to 1000 nm, and more preferably between 590 to 750 nm, and most preferred between 600 to 700 nm.

1. An ingestible device comprising: a capsule, where housed within the capsule are (1) a plurality of light sources, (2) a controller board, and (3) at least one battery; and where the light sources and the battery are connected to the controller board.

2. The controller board of claim 1, where the controller board acts as a switch to turn the device on or off.

3. The controller board of claim 2, where the controller board selects for the light sources to emit light continuously or intermittently.

4. The controller board of claim 3, where the controller board selects the wavelength of light emitted.

5. The controller board of claim 4, where the controller board selects for the intensity of light emitted.

6. The controller board of claim 5, where the controller board can be operated either manually or remotely.

7. The controller board of claim 1, where the controller board is programmable.

8. The controller board of claim 7 has a program installed, where said program controls the following functions: (a) on/off switch, (b) timer, (c) selecting for continuous light emission, (d) selecting for pulsed light emission, (e) selecting for the spectrum or wavelength of the light emitted, (f) selecting for the type of light emitted, and (g) the selecting for the intensity of light emitted.

9. Where said program of claim 8 is hardwired into the controller board.

10. Where said program of claim 8 is programmed into the controller board by wired or wireless communication.

11. The device of claim 1, where the light sources are arranged in arrays that allow for broad illumination.

12. The device of claim 1, where at least one wavelength of the light emitted is selected from the electromagnetic spectrum.

13. The device of claim 1, where at least one wavelength of the light emitted is selected from the visible light spectrum.

14. The device of claim 1, where at least one wavelength of the light emitted is any wavelength between 600 to 1000 nm.

15. The device of claim 1, where at least one wavelength of the light emitted is selected from the red-light or infra-red portion of the electromagnetic spectrum.

16. An ingestible device comprising a capsule, where housed within the capsule are (1) a plurality of light sources, (2) a controller board, and (3) at least one battery; and where the light sources and the battery are connected to the controller board; where the controller board is operated manually or remotely; where the controller board acts to turn the device on or off, to select for the light sources to emit light continuously or intermittently, to select for the wavelength of light emitted, and to select for the intensity of the light emitted; where the diodes are arranged in arrays to maximize illumination; and to select for the wavelength of light emitted.

17. The device of claim 16, where the wavelength of light emitted is selected from the electromagnetic spectrum.

18. The device of claim 16, where the wavelength emitted consists of any one of the following: blue, green, red, or near-infrared.

19. The device of claim 16, where the wavelength of light emitted is any wavelength between 600 to 1000 nm.

20. A method to provide light therapy to the gastrointestinal tract by ingesting the device of claim 16.

21. A method for enhancing the gastrointestinal microbiome by ingesting the device of claim 16.

22. A method for increasing the diversity of the microflora in the gastrointestinal tract by ingesting the device of claim 16.

23. A method for removing inflammatory mediators in the gastrointestinal tract by ingesting the device of claim 16.
